(19) United States
(12) Patent Application Publication
(10) Pub. No.: US 2015/0045381 A1
(43) Pub. Date: Feb. 12, 2015

(54) COMPOSITIONS AND METHODS FOR SUSTAINED ENERGY AND ENHANCED WEIGHT LOSS

(71) Applicant: Alfred A. Lamoureux, JR., Hoover, AL (US)

(72) Inventor: Alfred A. Lamoureux, JR., Hoover, AL (US)

(21) Appl. No.: 14/452,511

(22) Filed: Aug. 5, 2014

Related U.S. Application Data
(60) Provisional application No. 61/862,946, filed on Aug. 6, 2013.

(51) Int. Cl. A61K 31/51; A61K 31/197; A61K 31/198

(52) U.S. Cl. CPC A61K 31/51; A61K 31/198; A61K 31/197
USPC 514/276

(57) ABSTRACT

Disclosed are compositions and methods for sustained energy and enhanced weight loss. A composition for weight loss and sustained energy in a person comprises an effective amount of one or more B vitamins and an effective amount of one or more of lysine, leucine, and methionine. The composition may further comprises an effective amount of one or more of a carbohydrate, cobalmin, and carnitine. The composition may be in a form administrable to a person orally, by intravenous infusion, or by intramuscular injection. A method of losing weight and sustaining energy comprises administering, to a person, a composition comprising an effective amount of one or more B vitamins and an effective amount of one or more of lysine, leucine, and methionine. The method may further comprise administering an effective amount of at least one of a carbohydrate, cobalmin, and carnitine.

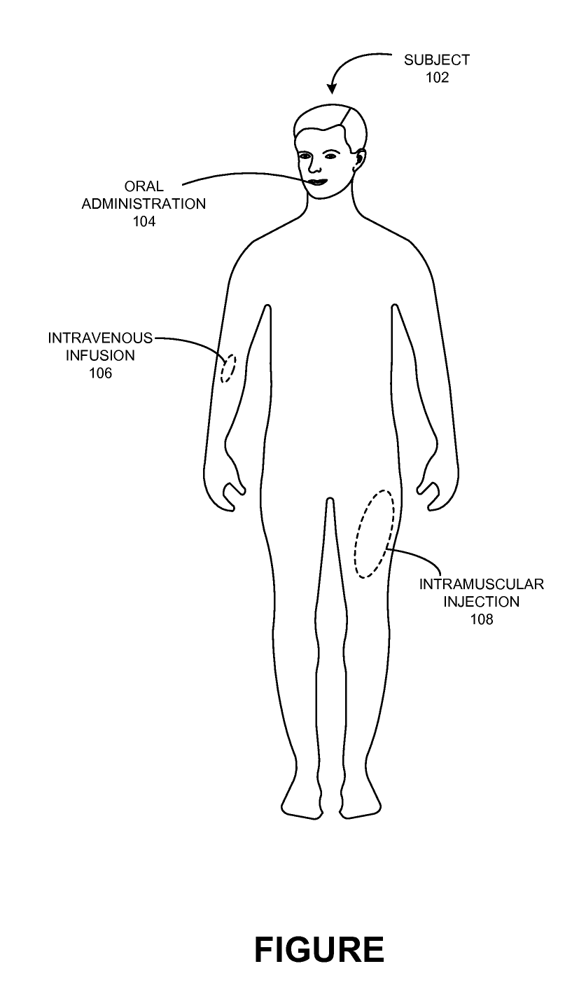

### CLAIM OF PRIORITY

This application is a non-provisional application of and claims priority to the US provisional application Ser. No. 61/862,946 tiled “COMPOSITIONS AND METHODS FOR SUSTAINED ENERGY AND ENHANCED WEIGHT LOSS” filed on Aug. 6, 2013.

### FIELD OF TECHNOLOGY

This disclosure relates generally to weight loss and, in one example embodiment, compositions and methods for sustained energy and enhanced weight loss.

### BACKGROUND

Weight gain results from caloric intake that is higher than the number of calories used in basal metabolism, and excess calories are stored as fat deposits. Weight is a leading risk factor for heart disease, hypertension, stroke, diabetes, and a variety of other conditions and diseases.

A customer may wish to achieve weight loss while sustaining or improving his/her energy level. The customer may be motivated to lose weight in order to mitigate health risks and/or social stigma associated with being overweight. The customer may therefore wish to utilize a composition containing multiple components understood to improve weight loss and/or energy level by enhancing lipid metabolism. However, the customer may not have the time or the knowledge to ensure a proper dose of the multiple components. In addition, the customer may desire a composition that can be administered orally (e.g. as a pill, a powder, a liquid, etc.) or parenterally (e.g. by intravenous infusion, by intramuscular injection, etc.)

Without committing to one particular physiological mechanism, certain B vitamins are understood to play a role in lipid metabolism and weight loss. In addition, the amino acids leucine, lysine, and methionine are understood to regulate lipid metabolism and aid in weight loss. Methionine, is understood to aid in the biosynthesis of choline, which may work synergistically with vitamin B-12. Both leucine and methionine are biosynthesized to produce carnitine, a compound involved in the breakdown of lipids. The customer may therefore desire a composition that provides one or more of the amino acids leucine, lysisne, and methionine as well as one or more B-vitamins.

### SUMMARY OF THE INVENTION

Disclosed are compositions and methods for sustained energy and enhanced weight loss. As disclosed herein, a composition for weight loss and sustained energy in a person comprises an effective amount of one or more B vitamins and an effective amount of one or more of lysine, leucine, and methionine. The composition may further comprises an effective amount of one or more of a carbohydrate, cobalmin, and carnitine. The composition may be in a form administrable to a person orally, by intravenous infusion, or by intramuscular injection.

A method of losing weight and sustaining energy comprises administering, to a person, a composition comprising an effective amount of one or more B vitamins and an effective amount of one or more of lysine, leucine, and methionine. The method may further comprise administering an effective amount of at least one of a carbohydrate, cobalmin, and carnitine. The method may further comprise administering the composition orally, through a peripheral vein by intravenous infusion, and/or by intramuscular injection.

The compositions and methods disclosed herein may be implemented in any means for achieving various aspects. Other features will be apparent from the accompanying drawings and from the detailed description that follows.

### BRIEF DESCRIPTION OF THE DRAWING

Example embodiments are illustrated by way of example and are not limited to the figures of the accompanying drawings, in which, like references indicate similar elements.

FIGURE is an illustrative diagram of routes by which the composition of the above embodiments may be administered to a subject, according to one or more embodiments.

Other features of the present embodiments will be apparent from the accompanying drawings and from the detailed description that follows.

### DETAILED DESCRIPTION

Disclosed are compositions and methods for sustained energy and enhanced weight loss. Although the present embodiments have been described with reference to specific example embodiments, it will be evident that various modifications and changes may be made to these embodiments without departing from the broader spirit and scope of the various embodiments. In addition, the components shown in the figures, their connections, couples, and relationships, and their functions, are meant to be exemplary only, and are not meant to limit the embodiments described herein.

In one or more embodiments, the invention provides a composition for weight loss and sustained or improved energy comprising an effective amount of at least one B vitamin (e.g. vitamin B-1, B-2, B-3, B-5, B-6 and B-12). Each of the B vitamins may be any form thereof familiar to one of ordinary skill in the art. For example, the composition may include a form of: vitamin B-1 (e.g. as thiamine), vitamin B-2 (e.g. as riboflavin, riboflavin-5′-phosphate, etc.), vitamin B-3 (e.g. as niacin, nicotinic acid, niacinamide, etc.), vitamin B-5 (e.g. as pantothenic acid, pantothenate, D-pantothenol, etc.), vitamin B-6 (e.g. as pyridoxal, pyridoxamine, pyridoxine, pyridoxine hydrochloride, etc.), and/or vitamin B-12 (cobalmin, methylcobalamin, cyanocobalamin, hydroxocobalamin, adenosylcobalamin, etc.). In addition, the composition comprises an effective amount of at least one of the amino acids lysine, leucine, methionine, and/or any forms or derivatives thereof familiar to one of ordinary skill in the art. Nutritional compositions typically administer an amino acid in the L-amino acid form (i.e. the biologically active form), although compositions comprising a mixture of amino acid forms where at least one form is an L-amino acid are within the scope of the embodiments disclosed herein.

B vitamins and the amino acids lysine, leucine, and methionine may function in lipid metabolism, may contribute to weight loss, and may sustain or enhance energy level. The novel combination of B vitamins (or forms thereof) and the aforementioned amino acids in a single composition for enhancing weight loss while sustaining or improving energy in a subject, is a preferred embodiment. Other embodiments may provide further additions to and forms of this composition.

In one or more embodiments, the composition may further comprise chromium. Chromium is a mineral present at trace amounts in an ordinary diet. It is understood to affect the appetite of a subject and is used in a number of weight loss regimens. Forms of chromium include chromium salts (such as chromium chloride) and chromium chelates (e.g. chromium nicotinate, chromium picolinate, etc.) and are within the scope of the embodiments disclosed herein.

In one or more embodiments, the composition may further comprise carnitine or any form thereof (e.g. L-carnitine, acetyl-L-carnitine, pantothenate, niacinamide, etc.). Carnitine is understood to be involved in the transport of fatty acids in the process of lipid metabolism. The amino acids lysine and methionine are biosynthesized to produce carnitine. L-carnitine is understood to be the biologically active form of carnitine.

In one or more embodiments, the composition may further comprise one or more carbohydrates. One example carbohydrate is inositol, which is understood to play a role in metabolism of lipid in the liver and may play a larger role in utilizing lipid stores as an energy souce. Other carbohydrates involved in lipid metabolism, energy level, and/or weight loss that are familiar to one of ordinary skill in the art are within the scope of the embodiments disclosed herein.

In one or more embodiments, the composition may further comprise choline. Choline is naturally biosynthesized and is available in a number of foods. Choline is understood to be involved in the metabolism of lipids in the liver and may play a larger role in utilizing lipid stores as an energy source. Forms of choline include choline salts (choline chloride, choline hydroxide, choline tartrate, etc.) and are within the scope of the embodiments disclosed herein.

In one or more embodiments, the composition may be for oral administration. The composition for oral administration may be a powder, a liquid, a gel, and/or another form for oral administration familiar to one of ordinary skill in the art. The composition for oral administration may include one or more flavoring agents. Examples of flavoring agents include a natural orange flavor, stevia extract, and/or any other flavoring agent familiar to one of ordinary skill in the art.

In one or more embodiments, the composition for oral administration comprises an effective amount of at least one B vitamin. When the composition for oral administration comprises vitamin B-1 (in any form), an example concentration of vitamin B-1 may be at or between 2 mg/mL and 50 mg/mL. When the composition for oral administration comprises vitamin B-2 (in any form), an example concentration of vitamin B-2 may be at or between 0.5 mg/mL and 15 mg/mL. When the composition for oral administration comprises vitamin B-3 (in any form), an example concentration of vitamin B-3 may be at or between 2 mg/mL and 50 mg/mL. When the composition for oral administration comprises vitamin B-5 (in any form), an example concentration of vitamin B-5 may be at or between 1 mg/mL and 25 mg/mL. When the composition for oral administration comprises vitamin B-6 (in any form), an example concentration of vitamin B-6 may be at or between 5 mg/mL and 60 mg/mL. When the composition for oral administration comprises vitamin B-12 (in any form), an example concentration of vitamin B-12 may be at or between 0.1 mg/mL and 5 mg/mL.

In one or more embodiments, the composition for oral administration also comprises an effective amount of at least one of the amino acids leucine, lysine, and methionine. When the composition for oral administration comprises leucine, an example concentration of leucine may be at or between 1 mg/mL and 25 mg/mL. When the composition for oral administration comprises lysine, an example concentration of lycine may be at or between 5 mg/mL and 50 mg/mL. When the composition for oral administration comprises methionine, an example concentration of methionine may be at or between 5 mg/mL and 50 mg/mL.

In one or more embodiments, the composition for oral administration may additionally comprise an effective amount of chromium (in any form). The chromium may be at a concentration at or between 10 ug/mL (micrograms per milliliter) and 800 ug/mL. In one or more embodiments, the composition for oral administration may additionally comprise an effective amount of carnitine (in any form). The carnitine may be at a concentration at or between 5 mg/mL and 50 mg/ml. In one or more embodiments, the composition for oral administration may additionally comprise an effective amount of a carbohydrate. For example, when the carbohydrate is inositol, it may be at a concentration of 10 mg/mL to 200 mg/mL. Other carbohydrates involved in lipid metabolism, energy level, and/or weight loss that are familiar to one of ordinary skill in the art are within the scope of the embodiments disclosed herein. In one or more embodiments, the composition for oral administration may additionally comprise an effective amount of choline (in any form). The choline may be at a concentration of 10 mg/mL to 200 mg/mL. In one or more embodiments, the composition for oral administration may additionally comprise other components understood by one of ordinary skill in the art to be helpful toward weight loss, energy level, and/or oral administration.

An example composition for oral administration that illustrates the present embodiment thereof is presented below. All concentrations are given as ranges of milligrams per milliliter (mg/mL) or micrograms per milliliter (mcg/mL).

| Component | Mass per milliliter |
|---|---|
| Thiamine | 2-50 mg |
| Riboflavin 5-Phosphate | 0.5-15 mg |
| Niacinamide | 2-50 mg |
| D-Pantothenol | 1-25 mg |
| Pyridoxine | 2-60 mg |
| Methylcobalamin | 0.01-5 mg |
| Leucine | 1-25 mg |
| Lysine | 5-50 mg |
| Methionine | 5-50 mg |
| Chromium | 10-800 mcg |
| Carnitine | 5-50 mg |
| Inositol | 10-200 mg |
| Choline Chloride | 10-200 mg |
| Natural Orange Flavor | 2-200 mg |
| Stevina Extract | 2-200 mg |

The components listed in the above example may be in any form previously described or familiar to one of ordinary skill in the art. Further, other embodiments of the composition may lack one or more of the components in the above example.

In one or more embodiments, the composition may be for parenteral administration (e.g. for administration by intravenous infusion or by intramuscular injection).

In one or more embodiments, when the composition is for intravenous infusion, concentrations of each component in the composition for intravenous infusion should ensure an effective dose of each component can be delivered to the subject while preventing irritation at the site of injection or other complications that can result from too high a concentration. Further, the composition for intravenous infusion may be a saline solution or combinable with a saline solution prior to administration. Still further, the composition for intravenous infusion should be at a pH around or between 6.6 and 7.2 or should be combinable with a buffered solution around or between 6.6 and 7.2 prior to administration.

In one or more embodiments, the composition for intravenous infusion comprises an effective amount of at least one B vitamin. Individual B vitamins may be at concentrations around or between 0.001% (percent weight) and 1%. Different B vitamins may be at different concentrations within the aforementioned range.

In one or more embodiments, the composition for intravenous infusion also comprises an effective amount of at least one of the amino acids leucine, lysine, and methionine. Individual amino acids may be at concentrations around or between 0.005% and 5%. Different amino acids may be at different concentrations within the aforementioned range.

In one or more embodiments, the composition for intravenous infusion may additionally comprise an effective amount of chromium (in any form). The chromium may be at a concentration around or between 0.000001% and 0.0005%. In one or more embodiments, the composition for intravenous infusion may additionally comprise an effective amount of carnitine (in any form). The carnitine may be at a concentration around or between 0.005% and 5%. In one or more embodiments, the composition for intravenous infusion may additionally comprise an effective amount of one or more carbohydrates, such as inositol, glycerol, dextrin or other carbohydrates involved in lipid metabolism, energy level, and/or weight loss that are familiar to one of ordinary skill in the art. The one or more carbohydrates may be at individual concentrations around or between 0.005% and 5%. In one or more embodiments, the composition for intravenous infusion may additionally comprise an effective amount of choline (in any form). The choline may be at a concentration at or between 0.005% and 5%. In one or more embodiments, the composition for intravenous infusion may additionally comprise other components understood by one of ordinary skill in the art to be helpful toward weight loss, energy level, and/or intravenous infusion.

In one or more embodiments, the composition may be in the form of an intramuscular injection (i.e. may be a composition for intramuscular injection). The component concentrations in the composition for intramuscular injection should ensure an effective dose of each component can be delivered to the subject while preventing irritation at the site of injection or other complications that can result from too high a concentration. The composition for intramuscular injection may additionally comprise an effective concentration of a local anesthetic. For example, the composition for intramuscular injection may comprise lidocaine at a concentration at or between 2 mg/mL and 40 mg/mL.

In one or more embodiments, the composition for intramuscular injection comprises an effective amount of at least one B vitamin. When the composition for intramuscular injection comprises vitamin B-1 (in any form), an example concentration of vitamin B-1 may be at or between 2 mg/mL and 50 mg/mL. When the composition for intramuscular injection comprises vitamin B-2 (in any form), an example concentration of vitamin B-2 may be at or between 0.5 mg/mL and 15 mg/mL. When the composition for intramuscular injection comprises vitamin B-3 (in any form), an example concentration of vitamin B-3 may be at or between 2 mg/mL and 50 mg/mL. When the composition for intramuscular injection comprises vitamin B-5 (in any form), an example concentration of vitamin B-5 may be at or between 1 mg/mL and 25 mg/mL. When the composition for intramuscular injection comprises vitamin B-6 (in any form), an example concentration of vitamin B-6 may be at or between 5 mg/mL and 60 mg/mL. When the composition for intramuscular injection comprises vitamin B-12 (in any form), an example concentration of vitamin B-12 may be at or between 0.1 mg/mL and 5 mg/mL.

In one or more embodiments, the composition for intramuscular injection also comprises an effective amount of at least one of the amino acids leucine, lysine, and methionine. When the composition for intramuscular injection comprises leucine, an example concentration of leucine may be at or between 1 mg/mL and 25 mg/mL. When the composition for intramuscular injection comprises lysine, an example concentration of lycine may be at or between 5 mg/mL and 50 mg/mL. When the composition for intramuscular injection comprises methionine, an example concentration of methionine may be at or between 5 mg/mL and 50 mg/mL.

In one or more embodiments, the composition for intramuscular injection may additionally comprise an effective amount of chromium (in any form). The chromium may be at a concentration at or between 10 ug/mL (micrograms per milliliter) and 800 ug/mL. In one or more embodiments, the composition for intramuscular injection may additionally comprise an effective amount of carnitine (in any form). The carnitine may be at a concentration at or between 5 mg/mL and 50 mg/ml. In one or more embodiments, the composition for intramuscular injection may additionally comprise an effective amount of a carbohydrate. For example, when the carbohydrate is inositol, it may be at a concentration of 10 mg/mL to 200 mg/mL. Other carbohydrates involved in lipid metabolism, energy level, and/or weight loss that are familiar to one of ordinary skill in the art are within the scope of the embodiments disclosed herein.

In one or more embodiments, the composition for intramuscular injection may additionally comprise an effective amount of choline (in any form). The choline may be at a concentration of 10 mg/mL to 200 mg/mL. In one or more embodiments, the composition for intramuscular injection may additionally comprise other components understood by one of ordinary skill in the art to be helpful toward weight loss, energy level, and/or intramuscular injection.

An example composition for intramuscular injection that illustrates the present embodiment thereof is presented below. All concentrations are given as ranges of milligrams per milliliter (mg/mL) or micrograms per milliliter (mcg/mL).

| Component | Mass per milliliter |
|---|---|
| Thiamine | 2-50 mg |
| Riboflavin 5-Phosphate | 0.5-15 mg |
| Niacinamide | 2-50 mg |
| D-Pantothenol | 1-25 mg |
| Pyridoxine | 2-60 mg |
| Methylcobalamin | 0.01-5 mg |
| Leucine | 1-25 mg |
| Lysine | 5-50 mg |
| Methionine | 5-50 mg |
| Chromium | 10-800 mcg |
| Carnitine | 5-50 mg |
| Inositol | 10-200 mg |
| Choline Chloride | 10-200 mg |
| Lidocaine | 2-40 mg |

The components listed in the above example may be in any form previously described or familiar to one of ordinary skill in the art. Further, other embodiments of the composition may lack one or more of the components in the above example.

In one or more embodiments, a method of weight loss and sustained energy in a subject comprises administering the composition of the above embodiments to the subject. The subject may be a child, an adult, or an elderly person of any weight. Further, the composition may be administered by a health care professional (e.g. a doctor, a nurse, etc.), by a non-professional (e.g. a family member or a friend of the subject), or by the subject himself.

FIGURE is an illustrative diagram of routes by which the composition of the above embodiments may be administered to a subject 102. In one or more embodiments, the method may comprise oral administration 104 of the composition. Alternatively, the method may comprise parenteral administration in the form of an intravenous infusion 106 or an intramuscular injection 108. The intravenous infusion 106 may be administered through any peripheral vein, and the composition administered should match the pH and salt concentrations in the blood. The intramuscular injection 108 may be administered by injection to the deltoid, the vastus lateralis, and/or the veentrogluteal or dorsogluteal muscles of the buttocks, or another muscle appropriate for intramuscual injection and familiar to one of ordinary skill in the art. Embodiments of the routes of administration are provided below.

In one or more embodiments, the method may comprise oral administration of one or more B vitamins. A weekly dose of each B vitamin may be around or between 10 mg and 5 g (grams). The method may additionally comprise oral administration of each of one or more amino acids at a weekly dose around or between 10 mg and 50 grams. The method may optionally further comprise oral administration of chromium at a weekly dose around or between 100 ug and 50 mg. The method may optionally further comprise oral administration of carnitine (in any form) at a weekly dose around or between 10 mg and 50 grams. The method may optionally further comprise oral administration of each of one or more carbohydrates (such as inositol) at a weekly dose around or between 50 mg and 200 g. The method may optionally further comprise oral administration of choline (in any form) at a weekly dose at or between 10 mg to 200 mg.

For example, the method may involve oral administration of the composition for oral administration of previously described embodiments at a volume in the tens, hundreds, or thousands of milliliters per week. In a further example, the composition for oral administration may be administered at mealtimes.

In one or more embodiments, the method may comprise parenteral administration (e.g. by intravenous infusion or by intramuscular injection) of one or more B vitamins. A weekly dose of each B vitamin may be around or between 1 mg and 200 mg. The method may additionally comprise parenteral administration of one or more amino acids. A weekly dose of each amino acid may be around or between 2 mg and 200 mg. The method may optionally further comprise parenteral administration of chromium at a weekly dose around or between 20 ug and 1 mg. The method may optionally further comprise parenteral administration of carnitine (in any form) at a weekly dose around or between 5 mg and 100 mg. The method may optionally further comprise parenteral administration of one or more carbohydrates (such as inositol) at a weekly dose around or between 10 mg and 200 mg. The method may optionally further comprise parenteral administration of choline (in any form) at a weekly dose at or between 10 mg to 200 mg.

For example, the method may involve administering the composition for intravenous infusion of previously described embodiments at a volume in the tens or hundreds of milliliters per week. In another example, the composition for intramuscular injection of previously described embodiments may be administered at a volume in the ones or tens of milliliters per week.

In one or more embodiments, the weekly doses of any of the embodiments of the methods disclosed herein may be administered as smaller doses throughout the week (e.g. twice a week, once a day, at every meal, etc.). In addition, the weekly doses may further depend on the weight, age, and/or gender of the subject.

The composition disclosed herein is safe to be taken regardless of the weight of the subject. The method of weight loss and sustained energy described in the above embodiments may be combined with additional weight loss regimens including exercise, dietary restriction, and weight loss treatments familiar to one of ordinary skill in the art.

A number of embodiments have been described. Nevertheless, it will be understood that various modifications may be made without departing from the spirit and scope of the claimed invention. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

What is claimed is:

1. A method of losing weight and sustaining energy, comprising:
administering, to a person, a composition comprising an effective amount of at least one B vitamin and an effective amount of at least one of lysine, leucine, and methionine.

2. The method of claim 1, further comprising administering an effective amount of at least one of a carbohydrate, cobalmin, and carnitine.

3. The method of claim 1, wherein the composition is administered orally.

4. The method of claim 1, wherein the composition is an infusion administered through a peripheral vein by intravenous infusion.

5. The composition of claim 1, wherein the composition is administered by intramuscular injection.

6. A composition for weight loss and sustained energy in a person, comprising:
an effective amount of at least one B vitamin; and
an effective amount of at least one of lysine, leucine, and methionine.

7. The composition of claim 1, further comprises an effective amount at least one of a carbohydrate, cobalmin, and carnitine.

8. The composition of claim 1, wherein the composition is in a form administrable, to a person, orally.

9. The composition of claim 1, wherein the composition is an infusion administrable, to a person, through a peripheral vein.

10. The composition of claim 1, wherein the composition is in a form administrable, to a person, through an intramuscular injection.
